(19) United States
(12) Patent Application Publication
(10) Pub. No.: US 2008/0014151 A1
(43) Pub. Date: Jan. 17, 2008

(54) Device and Method for Hand Washing

(76) Inventors: Susan K. Okuno-Jones, Buffalo Grove, IL (US); Jon M. Jones, Buffalo Grove, IL (US)

Correspondence Address: HARSHAW RESEARCH, INC., 210 W. TECUMSEH STREET, OTTAWA, KS, 66067, US

(21) Appl. No.: 11/847,482

(22) Filed: Aug. 30, 2007

Related U.S. Application Data
(63) Continuation-in-part of application No. 11/484,404, filed on Jul. 11, 2006, now abandoned.

(51) Int. Cl. A61K 8/33

(52) U.S. Cl. 424/10.3

(57) ABSTRACT

A device and method for washing a person's hands includes first and second cleaning agents having an indicator that is visually responsive to interaction of the cleaning agents. The cleaning agents may have different pH characteristics and the indicator is responsive to changes in these pH characteristics as the cleaning agents are mixed together. For example, the indicator may be blue in the presence of an unmixed cleaning agent but turns clear or some other color as the pH changes upon mixing with another cleaning agent. A dispenser includes separated chambers for holding respective cleaning agents, each chamber being associated with a pump and the pumps being associated to simultaneously expel respective cleaning agents. Thus, the cleaning agents may be dispensed onto a user's hands and, when the hands are rubbed together, produce the desired color change as the hands are thoroughly rubbed together.

### REFERENCE TO RELATED APPLICATIONS

The present application is a continuation in part of U.S. patent application Ser. No. 11/484,404, filed Jul. 11, 2006, and titled “Hand Washing System”, which is incorporated herein by reference.

### BACKGROUND OF THE INVENTION

This invention relates generally to hand washing solutions and, more particularly, to a device and method for washing a person's hands in which two cleaning agents may be dispensed and which include an indicator that is responsive to a thorough mixing of the cleaning agents.

It is well known that germs are often spread by a person's hands and this is especially the case when hands have not been washed thoroughly or frequently. Washing hands “properly” with running water and soap can reduce disease transmission and the spread of viruses and bacteria. The mechanical action of scrubbing loosens dirt and microbes on the hands and then soap, when brought into sufficient contact therewith kills the microbes and helps the water to wash them away. If hands are not washed properly, they can still be instruments for spreading germs.

Various products exist or proposals have been made in the prior art for promoting or indicating that a person's hands have been washed for a predetermined amount of time. Specifically, Patent Application Publication US 2005/0090414 proposes a pH indicator that may be dispensed into a person's hands to make contact with a pH altering compound. Although presumably effective, this proposal does not include a pair of cleaning agents each having opposed pH characteristics that interact to influence the other's pH and thereby influence a visual indicator. Further, Patent Application Publication US 2005/0049157 proposes use of a composition that changes color upon exposure to oxygen so as to indicate that the hands have been washed with the substance for a sufficient amount of time. Although assumably effective for their intended purposes, the existing products and proposals do not adequately insure that the hands have been thoroughly cleaned or do not provide the maximum cleaning effect.

Therefore, it would be desirable to have a device and method for washing a person's hands in which a color change occurs if two cleaning agents have been thoroughly mixed, whereby to indicate an adequate amount of scrubbing of the hands together. Further, it would be desirable to have a hand washing device and method having two independent cleaning agents that may be dispensed simultaneously from a dispenser having dual chambers. In addition, it would be desirable to have a hand washing method that may be used effectively and intuitively by children and adults.

### SUMMARY OF THE INVENTION

A device and method for washing a person's hands according to the present invention includes a first cleaning agent and a second cleaning agent. The second cleaning agent includes an indicator that is visually responsive to interaction with the first cleaning agent. More particularly, the cleaning agents produce a color change when they are thoroughly mixed together so as to indicate a sufficient amount of hand washing. Even though the color change occurs as a result of thorough mixing, it gives the impression to a user that the hands have been washed for a sufficient amount of time. The two cleaning agents have different pH characteristics and one of the cleaning agents also includes an indicator, the indicator being responsive to a change in the second pH as a result of thorough mixing of the two agents.

The hand washing method and device includes a dispenser having separate first and second chambers for containing the first and second cleaning agents, respectively. In addition, the dispenser includes first and second pumps connected to the first and second chambers, respectively, for dispensing respective cleaning agents. The dispenser also includes a pushplate for selectively actuating the pumps to dispense the cleaning agents. In other words, the two cleaning agents may be simultaneously dispensed, such as into a user's hand. In this way, a user may receive an amount of both agents into his hand and then mix them together as the hands are washed.

Therefore, a general object of this invention is to provide a device and method for washing a person's hands that includes two cleaning agents which, when thoroughly mixed together, produce a color change to indicate a sufficient amount of hand washing has taken place.

Another object of this invention is to provide a device and method, as aforesaid, in which the second of the two cleaning agents includes an indicator that is visually responsive to interaction with the first cleaning agent.

Still another object of this invention is to provide a device and method, as aforesaid, in which the pH of one cleaning agent is changed as a result of thorough mixing with the other cleaning agent.

Yet another object of this invention is to provide a device and method, as aforesaid, in which the pH characteristics of the cleaning agents are different and which are modified when the cleaning agents are mixed together.

A further object of this invention is to provide a device and method, as aforesaid, which includes a dispenser having separate dual chambers for holding the two cleaning agents apart from one another.

A still further object of this invention is to provide a device and method, as aforesaid, in which the dispenser includes a pair of pumps for simultaneously dispensing the two cleaning agents into a person's hand where they may be mixed together during a hand washing action.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a device for washing a person's hands according to a preferred embodiment of the present invention;

FIG. 2a is a side view of a dispenser of the hand washing device as in FIG. 1;

FIG. 2b is a sectional view taken along line 2b-2b of FIG. 2a;

FIG. 3 is an exploded view of the dispenser as in FIG. 1;

FIG. 4a is another side view of the dispenser as in FIG. 1, with the contents of the dispenser removed; and

FIG. 4b is a sectional view taken along line 4b-4b of FIG. 4a.

### DESCRIPTION OF THE PREFERRED EMBODIMENT

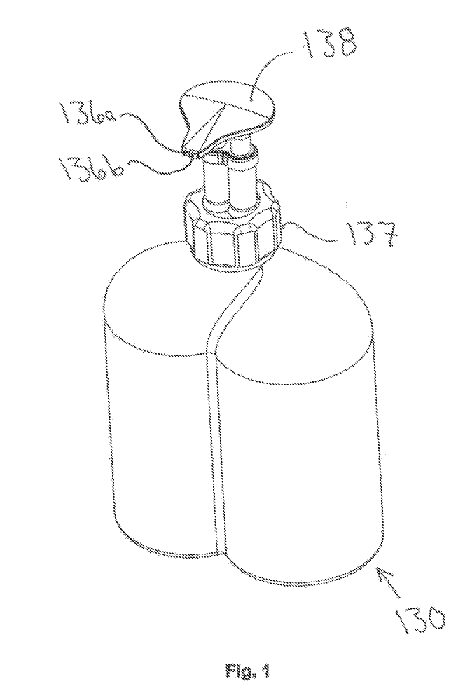

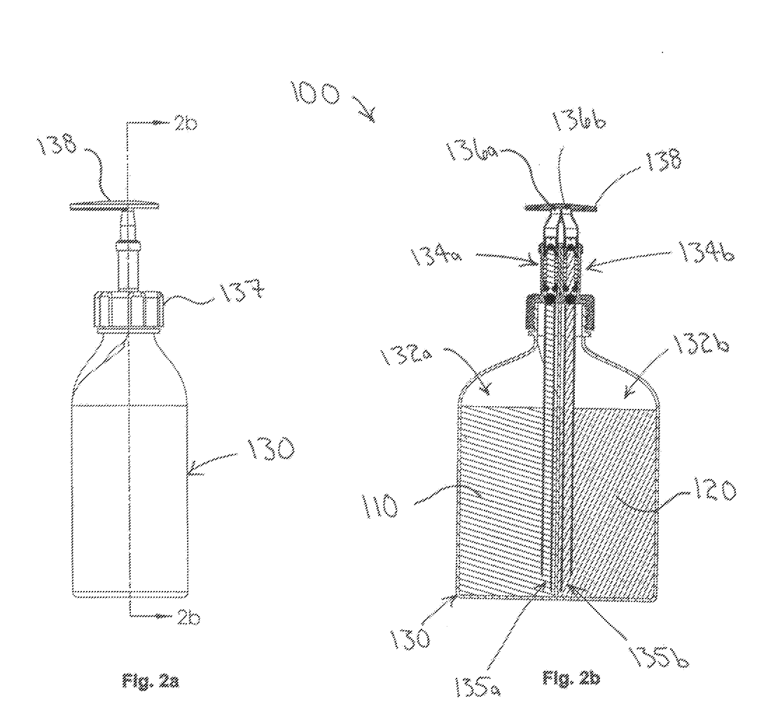

A device and method for washing a person's hands according to the present invention will now be described in detail with reference to FIGS. 1 through 4b of the accompanying drawings. More particularly, a hand washing device and method 100 according to the current invention includes a first cleaning agent 110 and a second cleaning agent 120 (FIG. 2b). The second cleaning agent 120 has an indicator that is visually responsive to interaction with the first cleaning agent 110.

The first cleaning agent 110 may have a first pH, the second cleaning agent may have a second pH, and the indicator may be visually responsive to a change in the second pH caused by thorough mixing of the first and second cleaning agents 110, 120. More particularly, either the first cleaning agent 110 or the second cleaning agent 120 may have a basic pH, the remaining cleaning agent 110, 120 may have an acidic pH, and the indicator may include a halochromic chemical compound capable of changing color (e.g., thymolphthalein, phenolphthalein, etc.). Both the first and second cleaning agents 110, 120 may be surfactant cleaning compounds, and the first and second cleaning agents 110, 120 may be anti-bacterial cleaning compounds, such as soap.

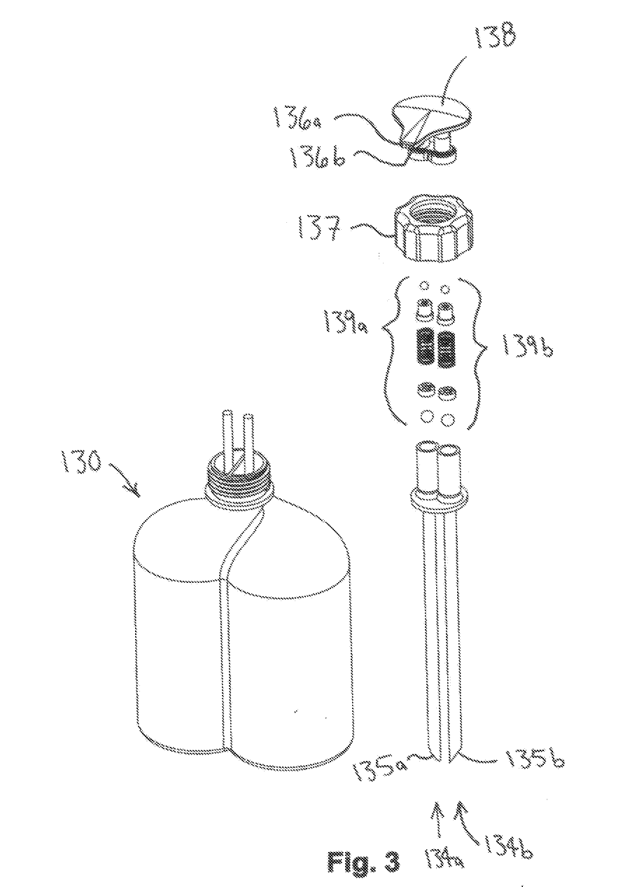

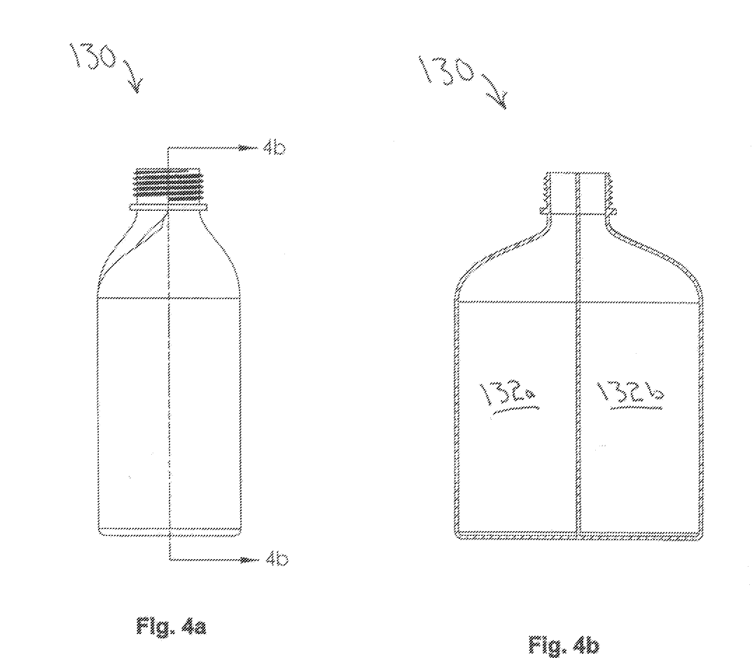

A dispenser 130 (FIGS. 1 through 4b) may have first and second pumps 134a, 134b and isolated first and second chambers 132a, 132b (FIGS. 2b and 4b) for selectively containing the first and second cleaning agents 110, 120 (FIG. 2b). The first pump 134a may have an inlet 135a inside the first chamber 132a and an outlet 136a outside the first chamber 132a, and the second pump 134b may have an inlet 135b inside the second chamber 132b and an outlet 136b outside the second chamber 132b (FIG. 2b). The first and second pumps 134a, 134b may include standard pump elements (springs, ball valves, etc.), which are collectively labeled 139a and 139b in FIG. 3. Means for associating the first pump 134a with the second pump 134b so that the first and second pumps 134a, 134b may be jointly actuated may also be included. More particularly, a pushplate 138 associating the first pump 134a with the second pump 134b may be included. The first pump 134a may be metered to selectively expel a predetermined amount of the first cleaning agent 110 onto a user's hands, and the second pump may be metered to selectively expel a predetermined amount of the second cleaning agent 120 onto a user's hands; this may be done by manipulating the standard pump elements 139a, 139b, for example. The predetermined amount of the first cleaning agent 110 may or may not be different from the predetermined amount of the second cleaning agent 120. Means for mounting the dispenser 130 on a wall may be included, such as a wall hook, eyelet, adhesive, hook and loop fasteners, etc.

In use, the first and second cleaning agents 110, 120 may be placed onto a user's hands, and the user may rub or scrub his hands with the first and second cleaning agents 110, 120. For purposes of demonstration, the first cleaning agent 110 may include citric acid, providing the first cleaning agent 110 with an acidic pH, the second cleaning agent 120 may have a basic pH, and the indicator may include thymolphthalein. In this example, the thymolphthalein would be blue while in the basic second cleaning agent 120. As the second cleaning agent 120 is mixed with the acidic first cleaning agent 110, the pH of the second cleaning agent 120 would become more acidic, the pH of the first cleaning agent 110 would become more basic, and the thymolphthalein would change color from blue to clear. When the color changed from blue to clear, then, the user would know that the first and second cleaning agents 110, 120 had been thoroughly mixed and that his hands had been thoroughly washed. The user's hands may then be rinsed under water until the cleaning agents are completely removed from the hands.

If the dispenser 130 were used to place the first and second cleaning agents 110, 120 in the user's hands, the user would push the pushplate 138 to simultaneously actuate both the first and second pumps 134a, 134b. When actuated, the first pump 134a would move an amount of the first cleaning agent 110 from inside the first chamber 132a, through the first inlet 135a, and out the first outlet 136a; the second pump 134b would move an amount of the second cleaning agent 120 from inside the second chamber 132b, through the second inlet 135b, and out the first outlet 136b. The pushplate 138 may ensure that both pumps 134a, 134b work simultaneously and provide an appropriate amount of each cleaning agent 110, 120. If all of one cleaning agent 110, 120 is used before the other cleaning agent 110, 120, the remaining cleaning agent 110, 120 may be used in a traditional manner to clean with. In other words, though there will be no indication that thorough cleaning has occurred, the remaining cleaning agent 110, 120 can be used and will not be wasted. Alternately, additional cleaning agents 110, 120 may be refilled into the dispenser 130 by removing the lid 137 (FIG. 3).

It is understood that while certain forms of this invention have been illustrated and described, it is not limited thereto except insofar as such limitations are included in the following claims and allowable functional equivalents thereof.

What is claimed is as follows:

1. A method for washing a person's hands, comprising the steps of:
providing a first cleaning agent and a second cleaning agent having an indicator that is visually responsive to interaction with said first cleaning agent, wherein:
said first cleaning agent has a first pH;
said second cleaning agent has a second pH; and
said indicator is responsive to a change in said second pH caused by thorough mixing of said first and second cleaning agents;
placing said first and second cleaning agents onto the person's hands;
rubbing the person's hands together whereby to mix said first and second cleaning agents together;
continuing to rub the person's hands together until said indicator changes to a predetermined color indicative of a thorough mixing of said first and second cleaning agents.

2. The method for washing a person's hands as in claim 1 further comprising the step of rinsing the person's hands with water after said indicator has changed to said predetermined color until said first and second cleaning agents are completely removed therefrom.

3. The method for washing a person's hands as in claim 1, wherein:
said indicator includes thymolphthalein;
said second pH is a basic pH; and
said first pH is an acidic pH.

4. The method for washing a person's hands as in claim 3, wherein;
said first cleaning agent includes citric acid; and
said citric acid provides said acidic pH.

5. The method for washing a person's hands as in claim 1, wherein:
one of said first cleaning agent and said second cleaning agent has a basic pH; and
another of said first cleaning agent and said second cleaning agent has an acidic pH.

6. The method for washing a person's hands as in claim 1, wherein said indicator is a halochromic chemical compound.

7. The method for washing a person's hands as in claim 6, wherein said indicator is selected from the group consisting of thymolphthalein and phenolphthalein.

8. The method for washing a person's hands as in claim 1, further comprising the steps of:
providing a dispenser having:
isolated first and second chambers, wherein said first cleaning agent is in said first chamber and said second cleaning agent is in said second chamber;
a first pump having an inlet inside said first chamber and an outlet outside said first chamber;
a second pump having an inlet inside said second chamber and an outlet outside said second chamber; and
a pushplate associating said first pump with said second pump and selectively actuating said first and second pumps.

9. The method for washing a person's hands as in claim 8 wherein the step of placing said first and second cleaning agents includes the step of pushing said pushplate to simultaneously actuate both said first and second pumps.

10. The method for washing a person's hands as in claim 8 further comprising the step of mounting said dispenser on a wall.

11. The method for washing a person's hands as in claim 8 further, comprising the step of rinsing the person's hands with water until the first and second cleaning agents are removed therefrom.

12. The method for washing a person's hands as in claim 8, wherein:
said indicator includes thymolphthalein;
said second pH is a basic pH; and
said first pH is an acidic pH.

13. The method for washing a person's hands as in claim 12, wherein;
said first cleaning agent includes citric acid; and
said citric acid provides said acidic pH.

14. The method for washing a person's hands as in claim 8, wherein:
one of said first cleaning agent and said second cleaning agent has a basic pH; and
another of said first cleaning agent and said second cleaning agent has an acidic pH.

15. The method for washing a person's hands as in claim 8, wherein:
said first pump is metered to selectively expel a predetermined amount of said first cleaning agent; and
said second pump is metered to selectively expel a predetermined amount of said second cleaning agent.

16. A method for washing a person's hands, comprising the steps of:
providing a first cleaning agent and a second cleaning agent having an indicator that is visually responsive to interaction with said first cleaning agent, wherein:
said first cleaning agent has a first pH;
said second cleaning agent has a second pH; and
said indicator is responsive to a change in said second pH caused by thorough mixing of said first and second cleaning agents;
providing a dispenser having:
isolated first and second chambers, wherein said first cleaning agent is in said first chamber and said second cleaning agent is in said second chamber;
a first pump having an inlet inside said first chamber and an outlet outside said first chamber;
a second pump having an inlet inside said second chamber and an outlet outside said second chamber; and
a pushplate associating said first pump with said second pump and selectively actuating said first and second pumps;
dispensing said first and second cleaning agents from said dispenser onto the person's hands;
rubbing the person's hands together whereby to mix said first and second cleaning agents together; and
continuing to rub the person's hands together until said indicator changes to a predetermined color indicative of a thorough mixing of said first and second cleaning agents.

17. The method for washing the hands of a person as in claim 16, further comprising the step of rinsing the person's hands with water after said indicator changes to a said predetermined color until said first and second cleaning agents are removed therefrom.

18. The method for washing the hands of a person as in claim 16, wherein:
one of said first cleaning agent and said second cleaning agent has a basic pH; and
another of said first cleaning agent and said second cleaning agent has an acidic pH;
said indicator is visually responsive to a change in said second pH caused by thorough mixing of said first and second cleaning agents.

19. The method for washing the hands of a person as in claim 16, wherein:
said first pump is metered to selectively expel a predetermined amount of said first cleaning agent onto the person's hands; and
said second pump is metered to selectively expel a predetermined amount of said second cleaning agent onto the user's hands.

20. The method for washing the hands of a person as in claim 16, further comprising the step of mounting said dispenser on a wall.
